SEQUENCE LISTING (13 sequences)

SEQ ID NO: 1 - protein, 5 aa, Artificial Sequence
  mass label
AVIVA

SEQ ID NO: 2 - protein, 5 aa, Artificial Sequence
  mass label
VVVGV

SEQ ID NO: 3 - protein, 5 aa, Artificial Sequence
  mass label
IIVAG

SEQ ID NO: 4 - protein, 5 aa, Artificial Sequence
  mass label
GGGKK

SEQ ID NO: 5 - protein, 5 aa, Artificial Sequence
  mass label
AAVIC

SEQ ID NO: 6 - protein, 5 aa, Artificial Sequence
  mass label
VGIAI

SEQ ID NO: 7 - protein, 5 aa, Artificial Sequence
  mass label
AIAVC

SEQ ID NO: 8 - protein, 13 aa, Artificial Sequence
  mass reporter
  DISULFID (9)..(11): The disulfide bond is located in-between a C-terminal and N-terminal peptide as a connecting linkage
  DISULFID (9)..(11): The disulfide bond is located in-between a C-terminal and N-terminal peptide as a connecting linkage.
IGMTSRYFCXCYR

SEQ ID NO: 9 - protein, 19 aa, Artificial Sequence
  mass reporter
  DISULFID (9)..(11): The disulfide bond is located in-between a C-terminal and N-terminal peptide as a connecting linkage
IGMTSRYFCXCFYRSTMGY

SEQ ID NO: 10 - protein, 9 aa, Artificial Sequence
  mass reporter
IGMTSRYFC

SEQ ID NO: 11 - protein, 9 aa, Artificial Sequence
  mass reporter
YGMTSRYFC

SEQ ID NO: 12 - protein, 9 aa, Artificial Sequence
  mass reporter
IGMGSRYFC

SEQ ID NO: 13 - protein, 9 aa, Artificial Sequence
  mass reporter
GGMTSRYFC

(19) United States
(12) Patent Application Publication
(10) Pub. No.: US 2023/0384314 A1
(43) Pub. Date: Nov. 30, 2023

(54) Oxidative Mass Labeling

(71) Applicant: LMX MedTech LLC, Bloomfield, MI (US)

(72) Inventors: Michael J. Pugia, Pagosa Springs, CO (US); Jason M. Kulick, South Bend, IN (US)

(21) Appl. No.: 18/249,095

(22) PCT Filed: Oct. 18, 2021
(86) PCT No.: PCT/US21/55402

§ 371 Date: Apr. 14, 2023

Related U.S. Application Data
(60) Provisional application No. 63/092,860, filed on Oct. 16, 2020.

(51) Int. Cl. G01N 33/58

(52) U.S. Cl. CPC G01N 33/581; G01N 33/582; G01N 33/587

(57) ABSTRACT

An electrochemical analyte detection method of cells and biomolecules that does not interfere with optical, genetic, or mass spectrometric detection allows reagents to be added simultaneously. Additional reagents can be included as mass reporters for identification of an analyte and measuring analyte integrity. Analyte integrity and identity allow electronic coupling of electrochemical analyte results to additional optical, genetic, or mass spectrometric results for the analyte. Reagents for mass reporters, electrochemical, and mass spectrometric detection can be added simultaneously to analyte detection microwells with size exclusion filters used for electrochemical signal generation and affinity agents for capturing mass reporters and analytes.

### CROSS REFERENCE TO RELATED APPLICATIONS

This application is the United States national phase of International Application No. PCT/US21/55402 filed Oct. 18, 2021, and claims priority to U.S. Provisional Patent Application No. 63/092,860, filed Oct. 16, 2020, the disclosures of which are hereby incorporated by reference in their entireties.

### SEQUENCE LISTING

The instant application contains a Sequence Listing which has been submitted in .txt format via EFS-Web and is hereby incorporated by reference in its entirety. Said txt copy is created on Oct. 18, 2021, named “Sequence Listing 95062105428 ST25”, and is 2,266 bytes in size.

### BACKGROUND OF THE INVENTION

### Field of the Invention

The present disclosure relates to a method for bio-analysis of complex samples and enabling biomolecule detection using a combination of electrochemical detection electronically coupled with additional downstream methods including optical detection, polymerase chain reaction (PCR) methods, gene sequencing methods, immunoassays, and mass spectrometric (MS) detection technologies as additional analysis. The detection of biomolecule and cells in whole blood, serum, plasma, urine, wound fluid, bronchial lavage, and other complex biological samples is improved in the disclosed. This disclosure expands the compatibility of electrochemical detection with other bio-analysis methods.

### Description of Related Art

Bio-analysis by combining results from multiple methodologies, namely electrochemical, optical, imaging, genetic, and mass spectrometric analysis, is becoming increasing more common for measurements of complex in-vitro, cell and tissue samples. Additionally, new mass spectrometric (MS) methods using mass labels to perform biomarker analysis are becoming increasingly more competitive and common, with noted ability to analyze rare biomolecules of interest at picomolar (pM) sensitivity. Such new methods lead to discovery of new masses, offering of high sample through-put, improved specificity, and/or multiplexed detection of multiple analytes in a single analysis.

New mass spectrometric (MS) methods using mass labeling are used commonly, such as the Isotope-Coded Affinity Tags (ICAT) method, Tag for Relative and Absolute Quantitation (iTRAQ), and Tandem Mass Tag (TMT) for mass spectrometric proteomic analysis. These methods of attaching a mass label to the biomolecule to be measured all suffer from contamination, expansive reagents, the use of isotopes as labels, and overlapping masses. These methods are also limited by the size and fragmentation of the analyte to be measured. Utilizing mass labels in mass spectrometric immunoassays (MS-IA) has improved the analysis by eliminating the need to purify the biomolecule before the analysis, by eliminating the need to chemically attach the mass label to the biomolecule, by eliminating the need for trypsin to fragment biomolecules, and the need for isotope labeling. A mass labeling approach for MS-IA utilizing a metal attached to antibodies and detecting the presence of metal by MS has been successfully used after optical imaging of cells and tissues. (Bandura 2009, Lee 2008). However, this requires expensive and specialized mass analyzers for metal detection.

Recently, the Signal Ion Emission Reactive Releasee Amplification (SIERRA) method for MA-IA (as described in, for example, Pugia U.S. Pat. Nos. 10,809,264, 11,061,035, Analytical Chem 2016, 2019, 2021) was developed using releasable organic compounds as the mass label attached to the affinity agents. These mass labels can be detected on almost any mass spectrometer (MALDI, ESI-MS-MS, Triple Quad) and eliminate the issues noted above. An alteration agent, namely, an acid or reducing agent, is used to break a linkage bond and release the mass label from the antibody for mass analysis. Detection of biomolecules in cells was demonstrated after optical imaging of cells with little background for MS-IA from complex samples (as described in, for example, Pugia Anal Chem 2016, 2019).

It is highly desirable that a rapid point of care (POC) method, such as electrochemical immunoassays (EC-IA), be compatible with the additional methods such as mass spectrometric (MS), optical imaging, and polymerase chain reaction (PCR) methods. Added flexibility of allowing the sample to be analyzed multiple times with these separate methods provides additional information and allows for laboratory re-testing of an analyte captured from a sample at POC. The SIERRA MS-IA has been shown to be compatible with optical fluorescence labels and to be non-destructive to DNA and RNA, allowing polymerase chain reaction (PCR) methods (as described in, for example, Pugia Anal Chem 2016, 2019, 2021). However, the electrochemical immunoassays (EC-IA) had to be run separately as electrochemical reactions during the electrochemical detection method due to the potential damage to the SIERRA reagent.

Previously, Pugia US2018/0284124 demonstrated that electrochemical reduction can break cleavable X-Y linkages of SIERRA reagents and that oxidation reactions can cause addition of phenols and amines to aromatic groups, peptides, proteins, and other biomolecules. Horseradish peroxidase (HRP) or alkaline phosphatase (ALP) are commonly used for electrochemical immunoassays (EC-IA) and are known to cause addition of phenolic and phenyl-amines substrates to aromatic amino acids. For example, immunoassays based on ALP and HRP enzymes such as Tyramide Signal Amplification (TSA™) (Pugia WO2015184144) cause covalent attachment of phenols and phenyl amines of the substrates to the peptide tyrosine groups.

PCT/US2020/055931 (hereinafter the “IBRI PCT”), which is incorporated by reference in its entirety, has recently demonstrated a device format that could perform multiple analysis of biomolecules directly on complex samples using multiple analyte detection microwells for electrochemical detection of target analytes. The analyte detection microwell includes a size exclusion filter with one or more pores, an electrochemical detector, and affinity agents for target analyte capture and detection which operate under a low hydrodynamic force. The affinity agent for detection is attached to a reagent capable of generating an electrochemical label. The affinity agent for capture is attached to a reagent capable of binding a surface in the microwell. The electrochemical label is detected by a working electrode and a reference electrode placed in the microwell to measure the label formed by the affinity agent for detection. The format enables capture of biomolecules and immunoassay detection of captured biomolecules in a convenient format without user intervention, with the added benefit of being able to remove and store the analyte detection microwell for additional downstream analysis.

The IBRI PCT device can be used with reagents for affinity assays such as electrochemical immunoassay (EC-IA), optical imaging, polymerase chain reaction (PCR) methods, mass spectrometric immunoassays (MS-IA), and mass spectrometric proteomics methods of the biomolecules captured on the analyte detection microwell. Descriptions of these assays utilized may be found in Pugia, M. J. et al., “Multiplexed SIERRA Assay for the Culture-Free Detection of Gram-Negative and Gram-Positive Bacteria and Antimicrobial Resistance Genes” Anal Chem, 2021 (published after provisional application). In one example, polyclonal affinity reagents were used as a sandwich assay pair by placing an affinity label (biotin) on some of the polyclonal antibody and placing an electro-chemical generating catalyst on the remaining polyclonal antibody. This format allows the biomolecules to be immediately captured on the filtration membrane using neutravidin. For multiplexed analysis, the filtration membrane is divided into multiple micro-wells with the size exclusion filter bottom.

While technologies capable of performing additional analysis of detection of rare molecules and cells after POC analysis exist, the field still requires improvements of electrochemical analysis methods to be compatible with mass spectrometric (MS) detection and other methods to allow simultaneous addition of all reagents in one sample processing step, and to allow performing an electrochemical analysis prior to mass analysis. A solution to this problem is the subject of this invention.

### SUMMARY OF THE INVENTION

An object of a non-limiting embodiment of the present disclosure is to provide an electrochemical analyte detection method of target analytes wherein said detection methods do not interfere with downstream detection where reagents can be added simultaneously and used in downstream analysis. This allows additional reagents to be included as mass reporters serving analyte standards for measuring analyte and demonstrating analyte integrity and identity. Analyte integrity and identity are used to allow electronical coupling of electrochemical analyte detection to the downstream optical, genetic, or mass spectrometric results. Mass reporters and reagents for electrochemical are added simultaneously to specific analyte detection microwells, where an affinity agent can capture mass reporter, analytes, and reagents used for electrochemical signal generation. In non-limiting embodiments or examples, the affinity agent is additionally labeled with the label for optical detection or mass labels for mass spectrometric immunoassays.

In non-limiting embodiments, the reagents for electrochemical, mass reporters, optical, and mass spectrometric detection are processed together in a common electrochemical sensor microwell with a size exclusion filter, electrodes, and affinity agents for analyte capture reagent and analyte detection.

In non-limiting embodiments, affinity agents used for analyte capture allow the capture of cell, virus, particles or biomolecules as analytes.

In non-limiting embodiments, mass reporters of product and sample integrity are included and capable of producing analyte identity and integrity as indication of suitability of results for electronically electrochemical results coupling to downstream analysis results.

In non-limiting embodiments, downstream analysis methods include optical methods, genetic methods such as polymerase chain reaction (PCR), molecular probes and sequencing, immunoassays, and mass spectrometric (MS) detection technologies.

Further non-limiting embodiments or examples are set forth in the following numbered clauses.

Clause 1: A method of analysis of complex samples comprising: introducing an affinity agent with an attached catalyst capable of forming an electrochemical signal; and measuring the electrochemical signal that is capable of being electronically coupled to results of downstream methods.

Clause 2: The method of clause 1, wherein the method contains no interference with optical, genetic, or mass spectrometric detection.

Clause 3: The method of any of clauses 1-2, wherein a mass reporter measures the integrity and identity of an analyte by downstream methods.

Clause 4: The method of any of clauses 1-3, further comprising processing reagents for downstream methods in a common electrochemical sensor microwell with a size exclusion filter, electrodes, and affinity agents for analyte capture reagent and analyte detection.

Clause 5: The method of any of clauses 1-4, wherein electrochemical signals are generated upon oxidation at non-reducing voltages of >-0.1 and do not produce acidic pH.

Clause 6: The method of any of clauses 1-5, wherein mass reporters are generated upon reduction at non-reducing voltages of <−0.1.

Clause 7: The method of any of clauses 1-6, wherein the mass reporter causes production of the optical, genetic, or mass spectrometric results as an analyte.

Clause 8: The method of any of clauses 1-7, wherein a mass reporter measurement allows electronical coupling of electrochemical analyte detection results to downstream methods results.

Clause 9: The method of any of clauses 1-8, further comprising introducing a catalyst, wherein the catalyst is an enzyme to generate the electrochemical signal.

Clause 10: The method of any of clauses 1-9, further comprising introducing a catalyst, wherein the catalyst is a nanoparticle to generate the electrochemical signal.

Clause 11: The method of any of clauses 1-10, wherein an affinity agent contains a fluorescent label for optical detection.

Clause 12: The method of any of clauses 1-11, further comprising detecting a mass, wherein the mass reporters comprises analytes and/or labels which can be peptides, proteins, genes or small biomolecules.

Clause 13: The method of any of clauses 1-12, further comprising introducing mass reporters to a microwell, wherein an analyte is released for detection by additional downstream methods analysis.

Clause 14: The method of any of clauses 1-13, wherein an affinity reagent captures mass reporters, cells, and analytes.

Clause 15: The method of any of clauses 1-14, further comprising analyzing captured analytes by downstream analysis methods, wherein the downstream analysis methods comprise downstream optical methods, mass spectrometric methods, immunoassay methods, and genetic methods.

These and other features and characteristics of the present disclosure, as well as the methods of operation and functions of the related elements of structures and the combination of parts, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 shows a schematic view of the principle used for electrochemical analysis of a sample in accordance with a non-limiting embodiment of the invention.

FIG. 2 shows the electrochemical response after addition of a sample by increased changes in current in accordance with a non-limiting embodiment of the invention.

FIG. 3 shows a schematic view of the principle used for mass spectrometric immunoassay analysis of a sample according to a non-limiting embodiment of the invention.

FIG. 4 shows a schematic view of the principle used for the mass reporter method for producing results indicating integrity and identity of the biomolecules captured in the analyte detection microwells according to a non-limiting embodiment of the invention.

FIG. 5 shows a schematic view of the principle used for the mass reporter method for producing results indicating integrity and identity of the multiple biomolecules captured in a set of multiple analyte detection microwells according to a non-limiting embodiment of the invention.

FIGS. 6A-6C show a scanning electrode microscope (SEM) view of the analyte detection microwell according to a non-limiting embodiment of the invention.

FIG. 7 shows a schematic view of a holder capable of holding and reacting fluids over the analyte detection microwells according to a non-limiting embodiment of the invention.

FIG. 8 shows a schematic view of a system for operation of the analyte detection microwells according to a non-limiting embodiment of the invention.

### DESCRIPTION OF THE INVENTION

For the purposes of promoting an understanding of the principles of the invention, reference will now be made to the embodiments illustrated in the drawings, wherein like reference numbers correspond to like or functionally equivalent elements, and specific language will be used to describe the same. It will nevertheless be understood that no limitation of the scope of the invention is thereby intended. Any alterations and further modifications in the described embodiments, and any further applications of the principles of the invention as described herein, are contemplated as would normally occur to one skilled in the art to which the invention relates. Certain embodiments of the invention are shown in detail, but some features that are well known, or that are not relevant to the present invention, may not be shown for the sake of conciseness and clarity.

For purposes of the description hereinafter, the terms “end,” “upper,” “lower,” “right,” “left,” “vertical,” “horizontal,” “top,” “bottom,” “lateral,” “longitudinal,” “forward,” “reverse” and derivatives thereof shall relate to the example(s) as oriented in the drawing figures. However, it is to be understood that the example(s) may assume various alternative variations and step sequences, except where expressly specified to the contrary. It is also to be understood that the specific example(s) illustrated in the attached drawings and described in the following specification are simply exemplary examples or aspects of the invention. Hence, the specific examples or aspects disclosed herein are not to be construed as limiting. Moreover, as used in the specification and the claims, the singular forms of terms include plural referents unless the context clearly dictates otherwise.

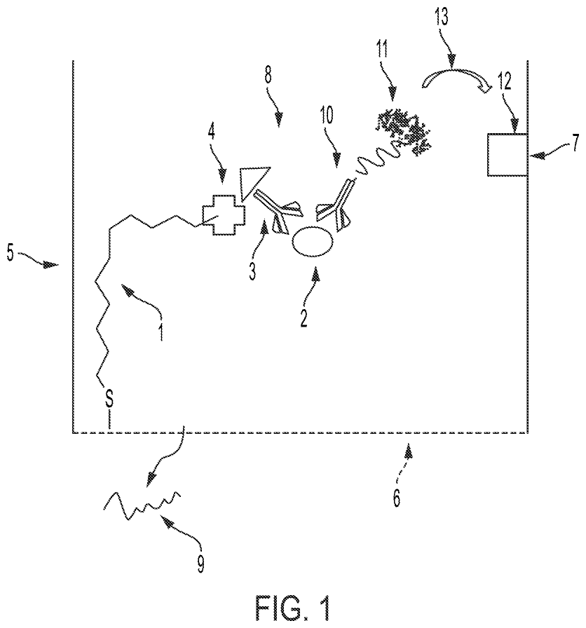

For purposes of the description hereinafter, an analyte detection microwell used for electrochemical detection of target analytes is described in accordance with the IBRI PCT (FIG. 1). The target analyte, analyte detection microwells, size exclusion filter, electrochemical detector, and affinity agents for a target analyte for capture and detection are defined as terms and examples in accordance with the IBRI PCT. The materials and methods described herein are useful with any of a broad variety of target analytes. The target analytes include a wide range of biomolecules and cells. In addition, the target analytes may comprise one or more target variants, as described hereinafter.

A non-limiting embodiment of the present disclosure is to provide an electrochemical analyte detection method of target analytes wherein said detection method does not interfere with downstream analysis methods. optical, genetic, or mass spectrometric detection. FIG. 1 shows a non-limiting embodiment of a electrochemical analyte detection method in accordance the IBRI PCT affinity agent reagent with an enzyme as a catalyst, namely, horseradish peroxidase or alkaline phosphatase, attached to the affinity agent and capable of generating an electrochemical signal which does not interfere with additional optical, genetic, or mass spectrometric detection methods. Other non-limiting examples of electrochemical signal generating reagents include other enzymes, proteases, and metal chelates capable of producing labels which are capable of being oxidized or reduced. Other non-limiting examples of optical, genetic, or mass spectrometric detection include polymerase chain reaction (PCR) immunoassay methods. Other non-limiting examples of electrochemical signal generating reagents include nanoparticles, metal particles, metal chelates, and organic molecules capable of being directly oxidized or reduced as electrochemical signals. Other non-limiting examples include adding additional electrochemical reagents such as conductive liquids, mediators, and others which can be added to improve the ability of the electrochemical signals to be measured. The affinity agent can be additionally labeled with the fluorescent label for optical detection. Optical fluorescent labels attached to affinity agents can be used additionally for signal generation as previously demonstrated (for example, in Pugia Anal Chem 2016 and 2019, which are incorporated by reference in their entireties).

The affinity agent can be additionally labeled with the mass labels for MS detection. In practice, the invention can make use of the same immunoassay reagent methods used for the electrochemical immunoassay (EC-IA) and SIERRA mass spectrometric immunoassays (MS-IA) as previously described as an example (Pugia Anal Chem 2017, 2019, 2021). These reagent methods can collect a sample and analyze the sample initially by reporting EC-IA results, which are discussed in Pugia et al. 63/006,833, 63/089,286, and 63/089,308 and incorporated herein by reference in their entireties. These reagent methods can electrochemically generate a signal as current in μA plotted against the voltage (V) for the electrochemical reporter captured by a high-affinity biotin onto a neutravidin linked to the size exclusion filter in a microwell with electrode.

The following FIGS. 1-8 contain like reference numbers which correspond to functional elements of the method and system for electrochemical detection of analytes wherein said detection methods do not interfere with optical, genetic, or mass spectrometric detection and reagent can be added simultaneously.

FIG. 1 shows a schematic view of the principle used for electrochemical analysis of a sample where a linkage arm (1) can capture an analyte (2) by an affinity agent (3) either directly attached to the linkage arm (1) or bound to the linkage arm (1) via a high-affinity label and capture agent (4). The linkage arm (1) is further attached to a microwell (5) with a size exclusion filter (6) on the bottom and with an electrode (7). The analyte (2), such as a cell or biomolecule, is captured by an affinity agent (3) after the addition of a sample (8) and not released as waste (9) through the size exclusion filter (6). A second affinity agent for target analyte detection (10) attached to a signal generating reagent (11) is added for generating an electrochemical signal (12). An electrochemical reagent (13) is added to the microwell (5), and the electrochemical signal (12) is produced and the analyte is measured with an electrode (7) placed in the microwell (5) and is converted into a measurement of electrochemical response.

Referring to FIG. 1, an analyte (2) is detected in an analyte detection microwell (5) by electrochemical detection of a generated signal (11). The affinity agent for capture is attached to the microwell by a linkage arm (1) directly or through a high-affinity capture. The affinity agent for target analyte capture (3) and the affinity agent for detection (10) attached to a signal generating reagent (11) produce an electrochemical signal (12) when the analyte (2) is captured after addition of the sample (8), removing waste (9) and after electrochemical reagent (13) is added to the microwell (5). Then, the electrochemical signal (12) is produced and measured with the electrode (7) in the microwell and converted to a measurement of the analyte.

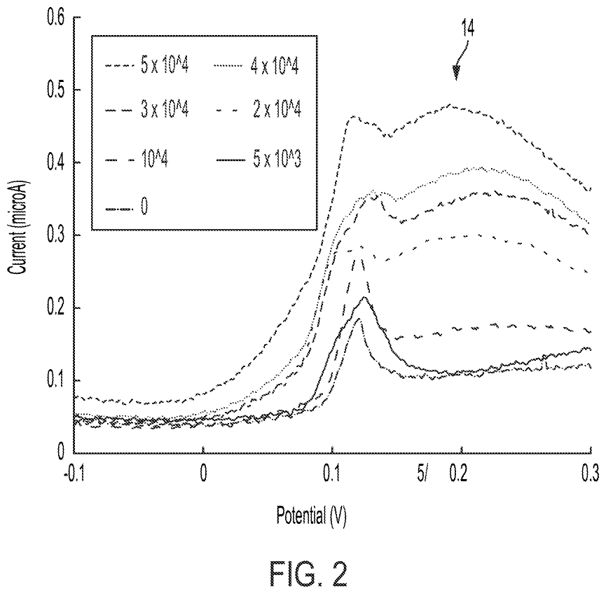

FIG. 2 shows the electrochemical response (14) after addition of a sample (8) caused by increased changes in electrochemical signal (12) for increasing numbers of P. aeruginosa (PA) cells measured after high-affinity capture of polyclonal antibody with biotin onto a neutravidin functionalized gold electrode surface and binding of a polyclonal antibody with alkaline phosphatase (ALP) and an electrochemical response (14) plotted as current signal versus the voltage and the responses are plotted for sample with 0, 5×10\hat{ }3, 10\hat{ }4, 2×10\hat{ }4, 3×10\hat{ }4, 4×10\hat{ }4 and 5×10\hat{ }4 PA cells/mL produced by p-aminophenol (pAP) generation. The electrochemical signal shown in FIG. 2 may be generated by the method as discussed above in FIG. 1.

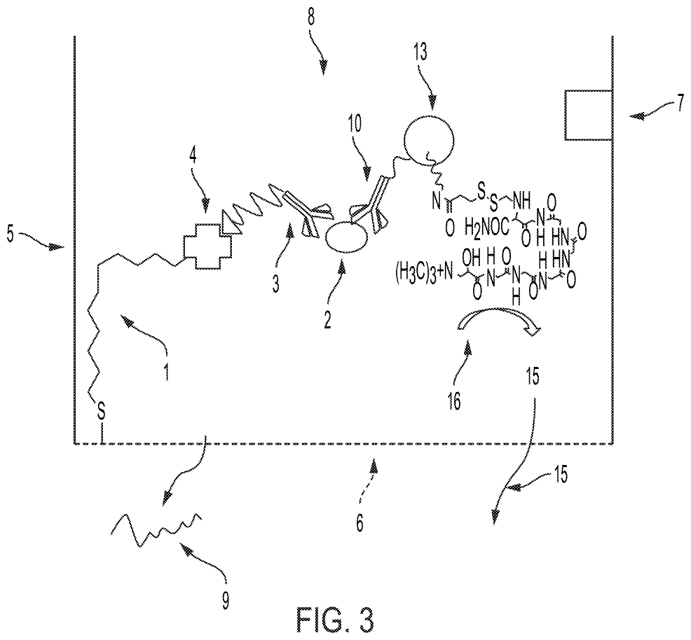

FIG. 3 shows a schematic view of a non-limiting embodiment of the invention for mass spectrometric immunoassay analysis of a sample where a linkage arm (1) can capture an analyte (2) by an affinity agent (3) either directly attached to the linkage arm (1) or bound to the linkage arm (1) via a high-affinity label and capture agent (4). The linkage arm (1) is further attached to a microwell (5) with a size exclusion filter (6) on the bottom and with an electrode (7). The analyte (2), such as a cell or biomolecule, is captured by an affinity agent (3) after the addition of sample (8) and is not released as waste (9) through the size exclusion filter (6). A second affinity agent (10) for a target analyte for detection is attached to a signal generating reagent (11) capable of generating a mass label (15) upon addition of an agent for releasing a mass label (15) for mass spectrometric analysis of the released mass label (15) after collected through the size exclusion filter (6). The mass label concentration measured is converted to a measurement of the analyte (2).

Referring to FIG. 3, a non-limiting embodiment of the present disclosure may include where an analyte (2) is additionally detected in the analyte detection microwell (5) by SIERRA mass spectrometric immunoassay (MS-IA) as previously described as an example (Pugia Anal Chem 2017, 2019, 2021). The method uses the same analyte detection microwell (5) with size exclusion filter (6) and electrode (7). The analyte is bound to the microwell (5) by a linkage arm (1) directly or through a high-affinity binding of a capture antibody. The SIERRA releasable mass labels (15) are bound to the analyte (2) through a detection antibody reagent (11) and released upon addition of an alteration agent (16) for releasing the free mass label (15), namely, an acid to break C—O bonds or a reducing chemical to break disulfide. The free mass label (15) is collected for mass spectrometric analysis through the size exclusion filter (6). The mass label concentration is measured is by a mass spectrometer and converted to a measurement of the analyte (2).

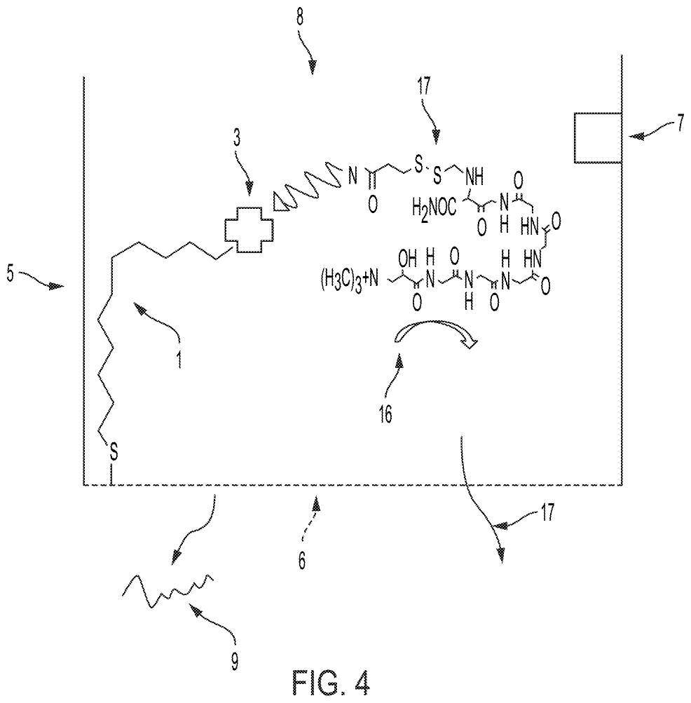

FIG. 4 shows a schematic view of a non-limiting embodiment of the invention to determine the identity of a microwell (5) by a mass reporter (17) captured by an affinity capture agent (4) or direct attachment to the linkage arm (1). The linkage arm (1) is further attached to a analyte detection microwell (5) with the size exclusion filter (6) and the electrochemical detector electrode (7). In some non-limiting embodiments, the analyte, such as a cell or biomolecule, is not captured by an affinity agent after the addition of the sample (8) and is released as waste (9) through the size exclusion filter (6). A mass label reporter is not released with waste (9) but remains in the microwell (5). The mass reporter (17) is released upon addition of agent for releasing the mass label (16) for detection by mass spectrometric analysis of the released mass reporter (17) which is collected through the size exclusion filter (6) and converted to a measurement of the identity of the microwell (5). In non-limiting examples, a cleavable bound is included in the mass reporter (17). In non-limiting examples, the mass reporter (17) label is freely released upon addition of an acid to break C—O bonds or reducing a S—S bond.

Referring to FIG. 4, a non-limiting embodiment of the present disclosure may include mass reporters that produce unique mass spectrometric (MS) fragment signals which are distinct from the mass of analytes or any mass labels used to make a measurement of the analyte. The mass reporter is measured and compared to expected values. Values within the expected range indicate analyte integrity. Analyte integrity is a lack of damage to the analyte as measured by a lack of damage to the mass reporter. An indication of integrity allows notification of suitable electrochemical results and suitable analytes for producing optical, genetic, and/or mass spectrometric analysis. In some non-limiting embodiments, the mass reporter shares common features of the analyte, such as peptides, carbohydrates, nucleic acids, and other biomolecules of the analyte, to allow integrity to be measured.

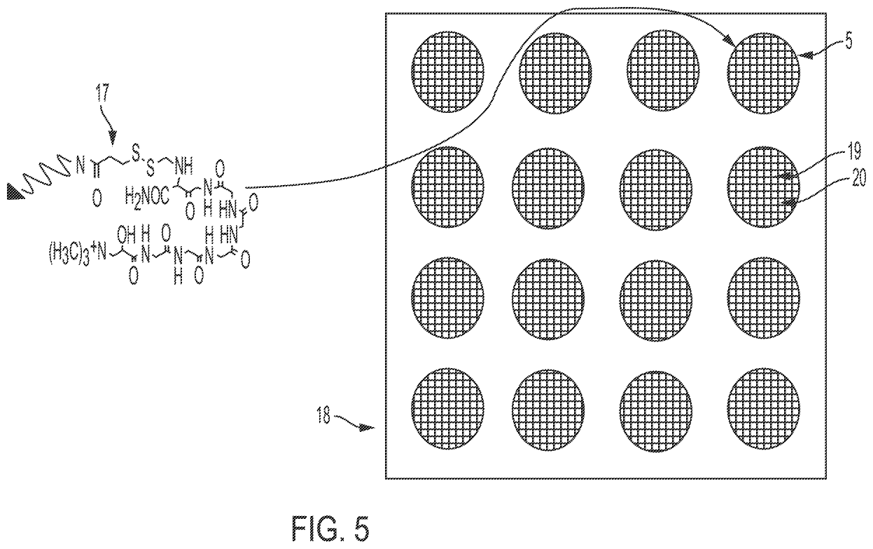

FIG. 5 illustrates a non-limiting embodiment of the present disclosure, whereby the mass reporter (17) is placed in a specific microwell (5) of a set of multiple microwells (18), such as a 4-by-4 array of microwells. This specific microwell (5) captures the mass reporter (17). The mass reporter (17) is freed and measured by a mass spectrometer to identify the microwell (5) from the rest of the set of multiple microwells (18). A second mass reporter (18) unique from the first mass reporter (17) is placed into a second microwell (19) and is used for reporting results indicating analyte integrity and identifies the second microwell (19). In non-limiting examples, the analyte (2) is additionally captured by an affinity agent (3) in the microwell (5), and the mass reporter (17) in the microwell (5) identifies the analyte (2) by a record of the identity of the affinity agent (3) added in the microwell (5) at the time of manufacturing. In non-limiting examples, the analyte (2) is captured by an affinity agent (3) and detected by a signal generating reagent (11), and the mass reporter (17) identifies the analyte (2) captured in the microwell (5) to allow linking electrochemical results to additional optical, genetic, and/or mass spectrometric analysis the microwells (5).

FIGS. 4 and 5 illustrate a non-limiting embodiment of the present disclosure, where a first mass reporter (17) and a second mass reporter (19) indicate integrity and identity of analytes (2) captured by affinity agents (3) in the first microwell (5) and the second microwell (20). In a non-limiting example, the method uses the microwells (5) with size exclusion filter (6) and electrodes (14) for analyte (2) capture and detection by an affinity agent (3) and signal generating reagents (11) for electrochemical or mass spectrometric mass label detection according to FIGS. 1 and 3. The analytes (2), signal generating reagents (11), and mass reporter (4) are captured in the first microwell (5) and the second microwell (20) after addition of the sample (8) and the waste (9) passes through the size exclusion filters (6). An electrochemical signal (12) is produced when the analytes (2) are measured with the electrode (7) in the first microwell (5) and the second microwell (20). Free mass reporters (17) and (19) are collected and identified by mass spectrometric analysis according to FIG. 4. The electrochemical signals (12) produced are used to select mass reporters (17) and (19) to be used to indicate integrity and identity of analytes (2) captured by affinity agents (3) in the first microwell (5) and the second microwell (20).

One or more other microwells (5) are used for only analyte detection by electrochemical or mass spectrometric mass label detection as shown in FIGS. 1, 2 and 3. Further additional microwells (5) can be used for detection of mass reporter labels and detection of analytes by electrochemical or mass spectrometric mass label detection. The mass reporter (16) is released after completion of electrochemical analysis and upon addition of the agent for releasing the mass reporter for mass spectrometric analysis which are collected through the size exclusion filter. Additionally, analysis of the analytes (2) released after collected through the size exclusion filter can be performed by optical, genetic, and/or mass spectrometric analysis. The MS reporter serves as a marker for microwell location from which analytes (2) were captured for detection by electrochemical signal and release for additional optical, genetic, and/or mass spectrometric analysis.

### Example 1: Method for Non-Interfering Oxidative Electrochemical Analysis for Simultaneous Sample Preparation for Mass Label Analysis

To demonstrate the invention, samples were simultaneously processed in an analyte detection microwell with mass reporters by the system, as shown in FIGS. 5, 6 and 7 with EC-IA reagents attached to alkaline phosphatase, and MS-IA reagents attached to SIERRA nanoparticles with cleavable S—S or C—O bond linkage arm (Pugia Anal Chem 2021). In this method, alkaline phosphatase is used to generate para-amino phenol as the electrochemical reporter from para-amino-phenyl phosphate. The analyte detection microwell allows using size exclusion filtration membranes and multiple individual microwells to be loaded with a sample to be assayed with the sensor microwells.

### Materials:

| | |
|---|---|
| Analyte | Analyte detection microwell of 110 and 200-μm diameters and 300 μm |
| detection | depths were made using standard microfabrication photolithography |
| microwell | techniques described below. Analyte detection microwells of 2000 μm |
| | diameters and 300 μm depths were alos fabricated under contract by Vishay |
| | (Shelton, CT) to design CAD produced by BioMEMS Diagnostic Inc. |
| Mass | SIERRA nanoparticles with S—S or C—O bond linkage arms were produced |
| labels | according to procedures provided in literature (Pugia Anal Chem 2021). The |
| | mass labels used were peptides. Nanoparticles with C—O bond linkage arms |
| | utilized betaine-Ala-Val-Ile-Val-Ala (AA-5), betaine-Val-Val-Val-Gly-Val |
| | (VV-5), betaine-Ile-Ile-Val-Ala-Gly (IG-5), and betaine-Gly-Gly-Gly-Lys- |
| | Lys (GL-5) as mass labels (15). Nanoparticles with S—S bond linkage arms |
| | utilized carnitine-Ala-Ala-Val-Iso-Cys (AC-5) as mass labels (15). The |
| | peptides betaine-Val-Gly-Ile-Al-Ile (VI-5) and carnitine-Ala-Iso-Ala-Val- |
| | Cys (AC-5.2) were used as internal standards. All mass label and reporters |
| | are resolvable as separate fragmentation signals in the MS-MS spectra when |
| | measured (Pugia Anal Chem 2021). |
| Mass | Mass reporters for protein analytes were tested using synthetic peptides (see |
| Reporters | Table 1). Mass reporters were produced with biotin and a -S—S- cleavable |
| | bond in Biotin Iie-Gly-Met-Thr-Ser-Arg-Tyr-Phe-Cys-S—S-Cys-Tyr-Arg |
| | (abbr. IC9-2B-S—S-RC-3-) and Biotin Iie-Gly-Met-Thr-Ser-Arg-Tyr-Phe- |
| | Cys-S—S-Cys-Phe-Tyr-Arg-Ser-Thr-Met-Gly-Tyr (abbr IC9-2B-S—S-YC-9) |
| | (Celtek LTD) Mass reporters for small molecule analytes were tested using |
| | synthetic homocysteine (Sigma Aldrich) Mass reporters for genes analytes |
| | were tested using synthetic RNA and DNA oligo with thiol modifiers |
| | (Integrated DNA technologies Inc). |
| Signal | The signal generating enzyme alkaline phosphatase (ALP), as well as gold |
| generating | nanoparticle (NP) of 5 to 100 nm diameter (Sigma Aldrich) were uses a |
| reagent (11) | examples of as electrochemical catalyst |
| Electrochemical | The ALP signal generating reagent used an electrochemical solution of 1.05 |
| solution | mM solution of p-amino-phenyl phosphate (pAPP, 3.0 mg, MW 189) in 100 |
| | mM TRIS, 600 mM NaCl, and 5 μM MgCl2 adjusted to pH 9.0. The NP |
| | signal generating reagent used an electrochemical solution of 5 mM |
| | potassium ferricyanide, 5 mM potassium ferrocyanide, 100 mM Tris-HCl pH |
| | 8, and 0.2% Tween-20. |
| Capture | Polyclonal antibodies recognizing S. aureus (Thermo Fisher Scientific), E. |
| antibodies | coli (MyBioSource, San Diego, CA, USA), K. pneumoniae (Thermo Fisher |
| | Scientific) and P. aeruginosa (Abcam, Cambridge, UK) were conjugated to |
| | biotin-PEG4 using EZ-Link NHS-conjugation kits (Thermo Fisher |
| | Scientific). The resultant antibody conjugates were stored at 4° C. |
| Detection | Detection antibodies were polyclonal antibodies recognizing S. aureus |
| antibodies | (Thermo Fisher Scientific), E. coli (MyBioSource, San Diego, CA, USA), K. |
| | pneumoniae (Thermo Fisher Scientific) and P. aeruginosa (Abcam, |
| | Cambridge, UK) separately conjugated to alkaline phosphatase (ALP) or |
| | horse radish peroxidase (HRP) as catalyst (Thermo Fisher Scientific) or to |
| | gold nanoparticle (NP) of 5 to 100 nm diameter as catalyst (Sigma Aldrich). |
| | The resultant antibody conjugates were stored at 4° C. |

Unless otherwise noted, all other materials were purchased from Sigma Aldrich or Thermo Fisher Scientific.

Method of Making Microwell Sensors with High-Affinity Capture Surface

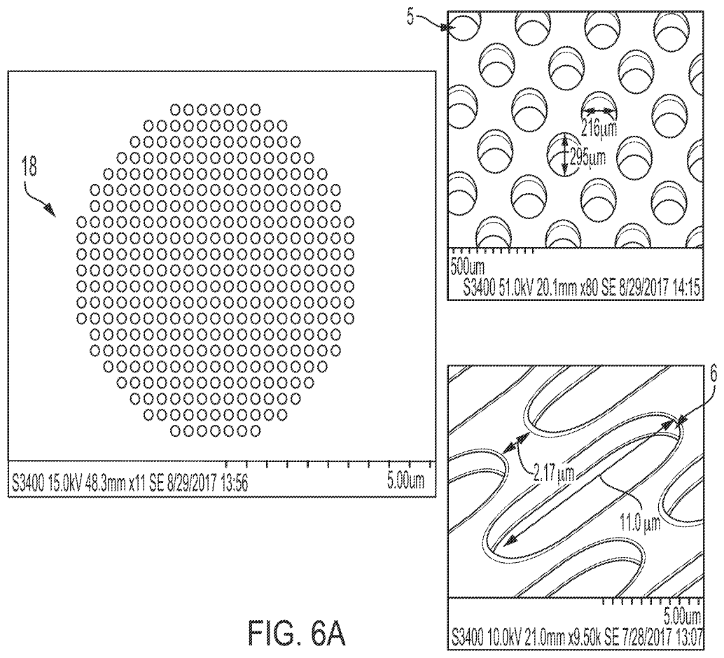

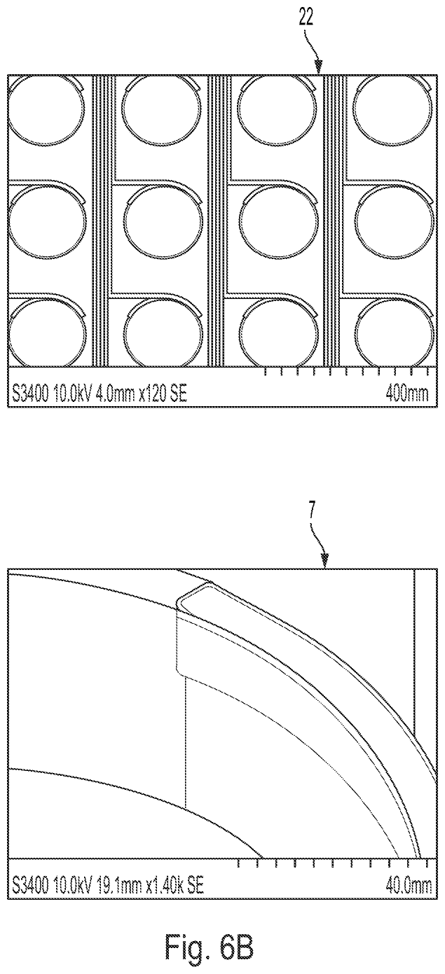

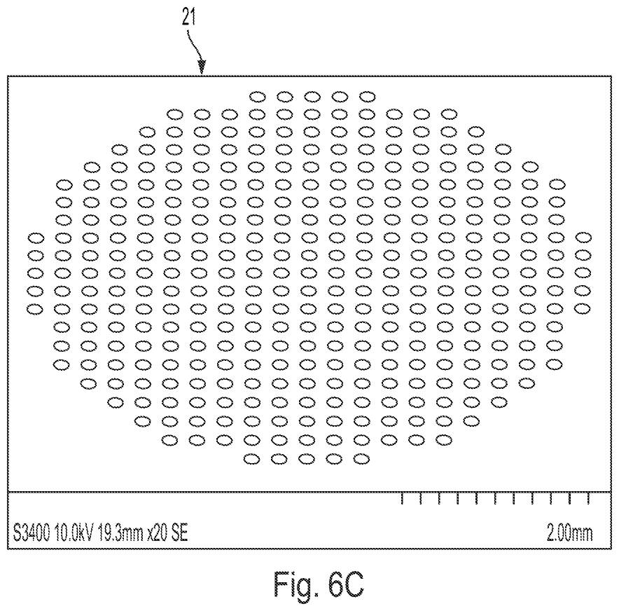

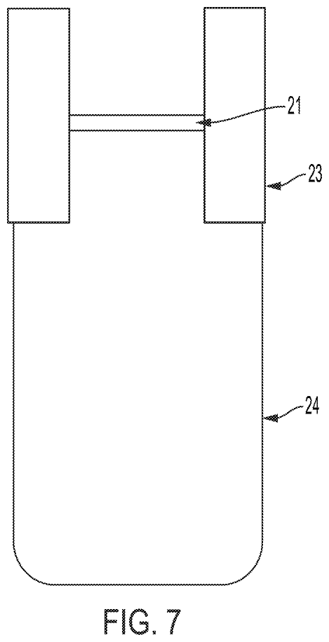

FIGS. 6A-6C illustrates a non-limiting embodiment of the present disclosure used for this example. This example method utilizes a set of multiple microwells (18) in a micro-filtration sensor (21), each with size exclusion filters (6) at the bottom (9.0×21.0 μm slot pores) with an overall pore space of 58 mm\hat{ }2 per well and sized to pass analytes not selectively bound to an affinity agent (3) in the microwell (5). (Methods as discussed according to FIGS. 1, 2 and 4). Additionally, each microwell (5) has an electrode (7) for routing the current lines (22) into any given microwell (5). This array of microwells (5) was fabricated in silicon dioxide (SiO₂) by double polishing the silicon wafer base substrate (300 μm thick) to make micro-filtration sensors (21) of 6.5 mm diameter with either 110 μm diameter microwells (5) or 200 μm diameter microwells (5). Microwells (5) had a 300 μm depth. The micro-filtration sensors (21) were connect to the analyzer using a holder (23) of 6.5 mm diameter and 10.9 mm depth and a waste containment area (24) shown in FIG. 7.

FIG. 7 shows a schematic view of a holder (23) capable of holding and reacting fluids over the analyte detection microwells (5) that also includes a waste collection area (24) that allows collection of release of the mass label through the analyte detection microwells (5) when hydrodynamic force is applied.

Micro-filtration sensors (21) with arrays of microwells (5) of either 110 or 200 μm diameters and 300 μm depths were made using standard microfabrication photolithography techniques with <0.1 μm dimensional tolerance. Microwells (5) were patterned with the arrays inside a 6.5-mm diameter of 35 mm 2 or the size of a conventional ELISA plate well. In brief, film layers (4 to 20 μm) of dense, high-quality thermal SiO₂ were patterned with a slotted pore (9.0×21.0 μm) grid serving as the size exclusion filter (6) by photolithography and dry etch processes. A 200-nm layer of gold was added to the size exclusion filter (6) by vapor deposition or coating of gold to serve as a gold electrode. A second layer of 300 μm thickness was made with silicon (110 or 200 μm wells) by the photolithography and dry etch processes to create a set of multiple microwells (18). The fabricated microwells layer was then mounted face up on the size exclusion filter (6), and the “top side” with the microwells (5) face up was further processed by etching electrode current lines (22) and filling with copper via electroplating and covering the lines with a protective layer to keep each of the microwells (5) readable.

The neutravidin was linked to the gold surface of the size exclusion filter (6) using the following functionalization procedure. The modification of the working electrode to functionalize the surface with neutravidin was performed by the 11-MUA, EDC, and HHSS method. This fabrication starts with dissolving 1.0 mM of 11-Mercapotundecanoic acid (11-MUA) into a 50 mM phosphate buffer solution at pH 10. Next, 150 μL of the solution is added to each well and allowed to sit overnight. The wells were washed with water 5 times and heated at 37° C. until dry. The terminal carboxylic groups (of 11-MUA) were then activated for 1 h by applying 150 μL of mM N-(3-dimethylaminopropyl)-N-ethylcarbodiimide hydrochloride (EDC) and 15 mM (N-hydroxy-succinimide ester (NHSS) in 50 mM phosphate buffer solution at pH 6.1. The sensor was washed with water 5 times and heated at 37 C until dry. Next, the surface of the working electrode was treated with 0.5 μL of neutravidin (Thermo fisher Prod. 31000) dissolved at 10.0 mg/mL into 50 mM phosphate buffer and reacted for 30 minutes to immobilize at 37° C. until dry. The sensor was washed with water 5 times and heated at 37 C until dry. In non-limiting examples, the neutravidin was replaced with alkaline phosphatase (1.7 mg/ml) and directly linked to the microwell.

After functionalization, the micro-filtration sensors were blocked with 200 μL solution of blocking buffer. The blocking buffer was made with 112.5 mL of water containing 10% Candor (Candor Bioscience, Cat. #110125), 3.18 g MOPSO, 1.50 g BSA (Bovine Serum Albumin (Fraction V), and 60 uL Proclin 200 and adjusted to pH 7.5 with 10 N sodium hydroxide and the buffer. After blocking overnight, the micro-filtration sensors were washed five times with 200 μL of TBS-T (Tris buffered saline with 0.05% Tween-20) and allowed to air dry.

### Methods to Process Sample, EC-IA, MS Labels, and MS Reporters in Sensor Microwells

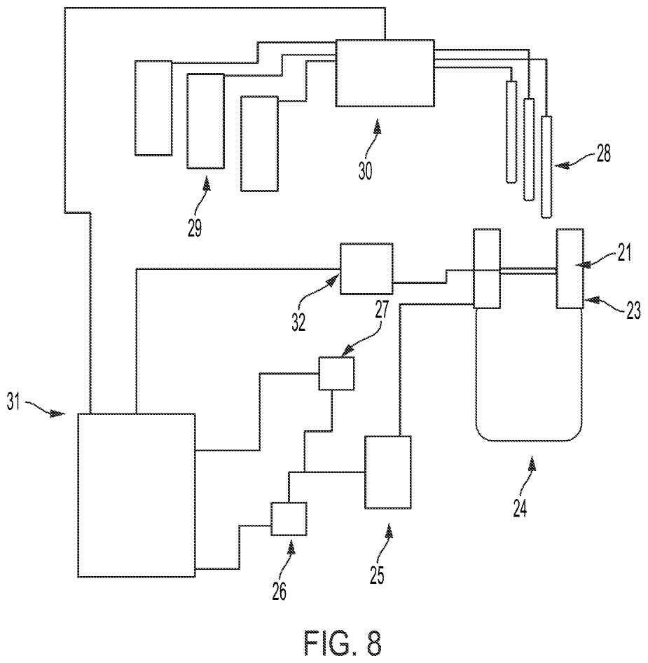

FIG. 8 shows a schematic view of a system, according to a non-limiting embodiment, for operation of the analyte detection microwells using a vacuum pump (25) connected to a vacuum pump motor driver (26) and a pressure sensor (27) as the hydrodynamic force for capturing cells and biomolecules from a sample in the analyte detection microwells (5) of the micro-filtration sensor (21). The micro-filtration sensor (21) is sealed to a sample and waste collection area (24) using a holder (23) for the micro-filtration sensor (21). The system includes fluidics for reactions with liquid reagents with dispensers positioned (28) over micro-filtration sensor (21) to feed liquid reagents (29) by programmable dispensing pumps (30). The vacuum pump motor driver (26), the pressure sensor (27), and the programmable dispensing pumps (30) are connected to a programmable controller board (31) used to monitor and regulate vacuum pressure for filtration by maintaining a user-defined pressure in the waste collection tube and dispensing liquid reagents by the programmable dispensing pumps (30). Voltages applied to electrodes in the analyte detection microwells (5) and current were read using electrochemical signals using a potentiostat (32) to read the micro-filtration sensor (21) and to measure voltage and current across working and reference/counter microelectrodes in each microwell.

FIG. 8 also illustrates a schematic of the system used in the example, as the hydrodynamic force for capturing cells and biomolecules from a sample in the microwells (5) of the micro-filtration sensor (21). The system uses vacuum filtration driven by an Arduino-based proportional-integral-derivative (PID) controller logic to maintain the desired pressure in the waste containment area (24). It also drives the sample and liquid reagent fluids through the size exclusion filters (6) in the microwells (5) of the micro-filtration sensor (21) held in a plastic format as a holder. The system serves as a sample processor by using a vacuum as the hydrodynamic force for capturing cells and/or biomolecules and analysis reagents onto the functionalized size exclusion filtration membranes in the microwells of the micro-filtration sensor. Negative pressure for filtration was provided by vacuum via the underside of the membrane.

The analyzer was built according to the schematic shown in FIG. 8 and included fluidic dispensers for addition of liquid reagents and electronics for detection of electrochemical signals via the electrode. An Arduino controller with a menu-driven program (Adafruit Industries, New York, NY, USA) was fitted on the Arduino, and a motor driver circuit board, a motor driver, and sensors were used to power, monitor, and regulate vacuum pressure for filtration (at 10-100 mbar negative pressure ±10%). An MPXV5050DP analog differential pressure sensor (Mouser Electronics, Mansfield, TX, USA) was used to measure the pressure in a conical 50-mL Falcon tube or 5-ml Eppendorf tube for sample and waste collection areas (24). An Arduino-based vacuum-driven fluidic control system including proportional-integral-derivative (PID) control maintains a user-defined pressure in the waste collection tube. The control loop also drives a DC diaphragm pump (22000.011, Boxer Pumps, Ottobeuren, Germany) through a DRV8838 brushed DC motor driver (Texas Instruments, Dallas, TX, USA) to evacuate air from waste collection areas (24). The pump and the pressure sensor were connected to the waste collection areas (24) using appropriate fluidic connectors (IDEX Health & Science, Oak Harbor, WA, USA). The liquid dispensing was controlled using the same Arduino controller and three peristaltic pumps with linear actuator motors to pump liquids into the sensor for reagent and sample delivery (100 uL±1%) through needles serving as dispensers (28).

Mass reporters (17) were made of peptide with amino acids common to protein analytes tested are shown in Table 1. Mass reporters (17) were also made to synthetic homocysteine as a small molecule analyte and synthetic DNA oligo with a thiol modifier as a genetic analytes. These mass reporters (17) could be biotinylated (see example IC9-2B) and additionally contain a cleavable bond like the S_S (See example IC9-2B-S-S-RC-3-4 and IC9-2B-S-S-YC-9). Mass reporters (17) with biotin and cleavable bond were added manually in a buffer to specific microwells with vacuum and washed with water and vacuum until dry.

EC-IA and MS-IA analysis used biotinylated antibody (3) for capture of analytes (2), alkaline phosphate (ALP) or nanoparticle labeled antibody as second affinity agent (10), signal generating reagent (11) for electrochemical detection, and SIERRA nanoparticles as signal generating reagent (13) for mass spectrometric detection of mass labels (15). Reagents were added manually to buffer complex sample analyte and incubated at 37° C. until adding to the analyte detection microwell with microsensors and neutravidin linked to the surface of the size exclusion filter. The antibodies used in this example are specific for the analyte detected. In this example, polyclonal antibodies recognizing S. aureus (Thermo Fisher Scientific), E. coli (MyBioSource, San Diego, CA, USA), K. pneumoniae (Thermo Fisher Scientific) and P. aeruginosa (Abcam, Cambridge, UK) were used. The analytes for each sample were bacterial lysates prepared at 5×10\hat{ }3 to 5×10\hat{ }4 cell/mL as described (Pugia Anal Chem 2021). The antibody reagents used for a different analyte are kept separated in different microwells.

Liquids were moved by the analyzer by using the program control logic controller (PID) to automate the additions and vacuum. Step 1) was drawing complex sample with antibodies down into microwells through turning the vacuum on; Step 2) was keeping complex sample with antibodies in microwells for incubation through turning the vacuum off; Step 3) was incubation of antigen and antibodies complex in sensor microwells for 5 minutes to allow the antigen affinity complex to be captured by the neutravidin attached to a size exclusion filtration membrane through a linkage arm; and Step 4) was addition and removal of wash solutions five times as 200 μL of TBS-T (Tris buffered saline with 0.05% Tween-20) to allow removal of unbound materials. The analyzer accomplished these steps, using the peristaltic pump with linear actuator motors to dispense the TBS-T wash and the vacuum to remove the TBS-T wash from the size exclusion filtration membrane.

The electrochemical response of the EC-IA was measured by the analyzer by using the program control logic controller (PID) to record the reading of the potentiostat after addition of 100 μL of electrochemical solution containing para-amino-phenyl phosphate (pAPP) as the electrochemical reagent (13) which allowed generating para-amino phenol (pAP) as the electrochemical signal (12) by using ALP as the signal generating agent (11) or electrochemical solution of 5 mM potassium ferricyanide, 5 mM potassium ferrocyanide, 100 mM Tris-HCl pH 8, and 0.2% Tween-20 by using the nanoparticle as the signal generating agent (11). The electrochemical signal generating is electrochemically measured with the potentiostat circuit board for calculating the response as a measurement of current changes vs voltage, as shown in FIG. 2. The signal can detected by or square wave voltammetry (SWV) as described in Pugia Anal Chem 2021. This was accomplished by application of a reference voltage and detecting the oxidized pAP generated using working/counter electrodes by a change in current measured across a working distance with a counter electrode. In another method, gold nanoparticle (NP) was used as the catalyst to generate a change in impedance as the generated electrochemical signal (as in Pugia, papers 1-3).

The analyzer used the potentiostat to read the sensor microwells and to measure and control voltages and current. The potentiostat circuit board allowed measurement of μAs across the working and reference/counter electrodes for −0.1 to 0.3 V. Each separate microwell was controlled through a multiplex board used in the potentiostat and the Arduino controller to deliver voltages and current results to the computer. The device was connected to a computer via a data storage card to provide all data from the Arduino for electrochemical analysis. The necessary hardware and electronics were fitted within a 12×21×6-inch case, including room for waste containment, three types of liquid reagents, the potentiostat, the microsensor and a small liquid crystal display (LCD) for the Arduino (PID).

### Demonstration of Detection by EC-IA, MS Labels and Mass Reporters

For demonstration of capture and detection of bacteria, S. aureus, E. coli or P. aeruginosa were grown in culture, and commercial antibodies for said cells were used. Cell lysates were prepared by addition of BPEP-II surfactant. To make the antibody complex for capture, a sample contained 100 μL of the lysate sample (0, 5, 10, 20, 30, and 40 thousand cells or lysate equivalent per assay) added to 48 μL of the biotinylated S. aureus, E. coli, or P. aeruginosa polyclonal antibodies (0.75 μg/assay) and 30 μL of the same polyclonal antibodies conjugated to ALP (1.50 μg/assay), SIERRA reagent (100 μg/assay) or mass reporter label (1.50 μg/assay) and incubated for 1 hour at room temperature.

The potentiostat circuit board allowed measurements for the EC-IA analysis from 3 μA to 100 nA current across the working and reference/counter microelectrodes for −0.1 to 0.3 V. A 333 and 33 pM of ALP produced average current change of 2.4 and 0.8 μA at 0.2 V in 5 minutes using an electrochemical reaction solution with 1 mM pAPP, 100 mM TRIS (3.1 g/200 mL), 600 mM NaCl (7.0 g/200 mL), and 5 μM MgCl (0.2 g/200 mL) adjusted to pH 9.0. The ALP activity is optimal in basic pH range of 8 to 9, and falls rapidly to little reactivity at pH 6.3. FIG. 2 shows the electrochemical signal generated as current in μA plotted against the voltage (V) for the immunoassay detection (EC-IA) directly on the binding surface for samples including either 0, 5, 20, 30, 40 or 50 thousand lysate equivalent of bacterial cells per assay. The immunoassay detection (EC-IA) directly on the binding surface achieved a quantitative bacterial immunoassay enumeration of cell counts across a range of 5,000 to 40,000 bacteria per sample, increasing concentration of the analyte.

For MS-IA analysis, an additional 100 μg of the SIERRA MS-IA reagent was added. For mass reporter analysis, an additional 1.5 μg of IC9-2B-S-S-RC-3-4 or IC9-2B-S-S-YC-9 was added as mass reporters. After incubated at room temperature, along with polyclonal antibodies conjugated to ALP (1.50 μg/assay) for the electrochemical response of the EC-IA was measured as described above (Also see Pugia Anal Chem 2021). MS-IA mass labels (15) and mass reporters (17) were then collected after washing with acid solutions for breaking the C—O bonds or reducing agents for breaking the S-S. FIG. 7 illustrates a non-limiting embodiment used to collect the mass labels and mass reporters from the micro-filtration sensor (21) into the sample collection area (24) by using the holder (23) for fluids and the micro-filtration sensor (21). The holder was connected to the micro-filtration sensor (21) and 5 mL microcentrifuge vial collection area (21 (e.g., a 5 mL Eppendorf tube), and hydrodynamic force was applied by centrifuging at 200×g for 2 min. This process allowed releasing and collection of mass label (15) or mass reporters to move through the micro-filtration sensor (6). The releases of the mass labels and/or mass reporters from the sensor surface were compared to expected values using a mass spectrometer. The mass label IC9-2B-S-S-RC-3-4 produced the RC-3-4 mass upon breakage of the —S-S— bond, and IC9-2B-S-S-YC-9 produced the YC-9 mass (Table 1).

Mass reporters (17) were made of synthetic homocysteine as a small molecule analyte and synthetic DNA oligo with a thiol modifier as a genetic analytes were released upon breakage of the —S—S— bond with reducing agents and were able to be detected by downstream analysis including optical methods, genetic methods such as polymerase chain reaction (PCR), molecular probes, immunoassays, and mass spectrometric (MS) detection technologies.

The concentration and spectra of released mass labels or mass reporters were determined using the LTQ-XL linear ion trap mass spectrometer (Thermo Fisher Scientific, Waltham, MA, USA) fitted with a Dionex Ultimate 3000 autosampler as previous described (Pugia Anal Chem 2019, 2021). For MS analysis, the read signal for the assay used a different mass label from the read signal for the assay and the reporter signal used as the internal reference standard, which was used for calibration against a plot of peak intensity ratio of mass labels to mass reporters versus the concentration of analyte in solution. Representative mass spectra for a MS label or MS reporters were observed for each label.

For C—O nanoparticles, calibration solutions were made containing either AA-5, VV-5, IG-5 or GL-5 at 52.6, 26.3, 13.15, 6.58, 3.29, and 1.644 nM with 52.6 nM for VI-5 internal standards. All were prepared in 10 mM ammonium acetate pH 5.5 buffer with 1:1 methanol. For S-S nanoparticles, the calibration solutions contained AC-5 across the sample levels with 25 nM for AC-5.2 internal standard. Blank solutions were also prepared in the same buffer with 52.6 nM VI-5 or 25 nM for AC-5.2 internal standards. The RML signals in the calibration solutions and samples were measured by MS/MS in centroid mode. MS/MS scans were used to monitor unique fragments for RML and internal standards to determine the mass label concentrations and correlate them to the number of bacterial cells using calibration curves.

Surprisingly, the EC-IA analysis across −0.1 to 0.3 V did not break S-S bonds or cause acid formation to break C—O bonds. Application of a 0.2 V over 5 minutes using the electrochemical reactions solution with 333 and 33 pM ALP did not release any detectable mass labels for either C—O or S-S nanoparticles. For comparison, 0.001% citrate release buffer (pH 5.2) caused complete and immediate release of the mass labels (15) and mass reporters (17) utilizing —C—O— cleavable linkages, and 100 μL of 5 mM TCEP caused release of the mass labels (15) and mass reporters (17) utilizing —S—S— cleavable linkages over 30 minutes. mass labels (15) and mass reporters (17) utilizing —S—S— cleavable mass reporters are generated upon reduction at non-reducing voltages of <−0.1. The concentrations of MS-IA mass labels (15) and mass reporters (17) released under breakage condition did not suffer damage due the EC-IA analysis. The expected mass labels (15) and mass reporters (17) masses and concentrations were observed. While not bound to mode of actions, it is believed the rapid cycle times of the EC-IA analysis due to small microwells are fast enough to avoid reducing and acid-forming conditions. Optical fluorescent labels and mass labels could be added simultaneously with reagents capable of generating an electrochemical signal. Genetic analysis of cells by polymerase chain reaction (PCR) methods could be performed after generating an electrochemical response without interference. The polymerase chain reaction (PCR) method was also not interfered with by analysis using optical or mass spectrometric labels.

### Demonstration of Further Non-Interference of Oxidative Electrochemical Methods

Mono-phosphate esters of substituted phenols, such para-amino-phenyl phosphate (pAPP), are highly reactant substrates for alkaline phosphatase (ALP). By way of example, multiple phosphate substrates have been studied including other aromatic rings and peptides but generally have no increased alkaline phosphatase (ALP) activity compared to substituted phenols, such as para-amino-phenol (pAP). The phenyl ring can be further substituted with a wide variety of organic atoms and groups along with any other group, such as fluorometric and optical labels, that do not increase alkaline phosphatase (ALP) activity.

Substituted phenols, such as para-amino-phenol (pAP) produced by ALP, are highly reactive, such that they undergo known auto-oxidation and dimerizing due to further oxidation by a second electron to reactive the para-quinone (pQ) species. These reactive pQ species are able to couple with aromatic phenols and amines in the Trinder's type reactions even under basic conditions (Pugia U.S. Pat. No. 5,362,633). These reactive pQ species are known to have potential to couple to a protein containing an attached phenolic residue, e.g., a tryrosine amino acid (Table 1).

To demonstrate a lack of interference for embodiment of the present disclosure, potential damage to peptides serving as examples of proteins as analytes for MS analysis were measured. The peptides contained Tryrosine (Tyr symbol) as a strong example of a receiving phenol for the para-quinone (pQ) (Table 1). After application of −0.1 to 0.2 V over 5 minutes using the electrochemical reactions solution with 333 and 33 pM ALP, no detectable changes to the mass labels were observed. A second voltage was applied at 0.1 V, and the pAP oxidization to para-quinone (pQ) occurred, as this is ideal voltage for the two-election oxidation with the loss of two electrons and two hydrogen from para-aminophenol (pAPP) to form para-quinone (pQ). Formation of this oxidation did not cause a coupling of the para-quinone (pQ) to the tryrosine of peptides in Table 1 or increase the mass or damage the mass label. Additionally, voltages greater than 0.1 V completely prevented the formation of para-quinone (pQ) to avoid any risk of damage to MS-IA mass labels (15) or mass reporters (17). The limit of how high this voltage needed to be was dependent on the electrode surface, working distance, and the composition of the electrochemical reaction solution; in this example, 0.2 V was sufficient to prevent damage.

TABLE 1
Mass reporter peptides with tryrosine amino acid residues

| Sequence* | Abbr. | Sequence* | C-terminus | MW (Da) |
|---|---|---|---|---|
| IGMTSRYFC | IC9-1 | Iie-Gly-Met-Thr-Ser-Arg-Tyr-Phe-Cys | NH2 | 1076.7 |
| Biotin- | IC9-2B | Biotin Iie-Gly-Met-Thr-Ser-Arg-Tyr- | NH2 | 1319.8 |
| IGMTSRYFC | | Phe-Cys | | |
| RYC | RC-3-4 | Arg-Tyr-Cys | NH2 | 355.4 |
| Biotin- | IC9-2B- | Biotin Iie-Gly-Met-Thr-Ser-Arg-Tyr- | NH2 | 1673.2 |
| IGMTSRYFC- | S-S- | Phe-Cys-S-S-Cys-Tyr-Arg | | |
| S-S-CYR | RC-3-4 | | | |
| YGMTSRYFC | YC-9 | Tyr-Gly-Met-Thr-Ser-Arg-Tyr-Phe-Cys | NH2 | 1027.3 |
| Biotin- | IC9-2B- | Biotin Iie-Gly-Met-Thr-Ser-Arg-Tyr- | NH2 | 2345.1 |
| IGMTSRYFC-S- | S-S- | Phe-Cys-S-S-Cys-Phe-Tyr-Arg-Ser- | | |
| S-CFYRSTMGY | YC-9 | Thr-Met-Gly-Tyr | | |
| IGMGSRYFC | IC9-3 | Iie-Gly-Met-Gly--Ser-Arg-Tyr-Phe- | NH2 | 1033.2 |
| | | Cys | | |
| YGMTSR*YFC | YC-9-2 | Tyr-Gly-Met-Thr-Ser-Arg*-Tyr-Phe- | NH2 | 1037.3 |
| | | Cys | | |
| GGMTSRYFC | GC-9 | Gly-Gly-Met-Thr-Ser-Arg-Tyr-Phe- | NH2 | 1021.4 |
| | | Cys | | |

### Demonstration of Electronic Coupling of Electrochemical and Mass Spectrometric Results

A method demonstrating that electrochemical results of the electrochemical immunoassays (EC-IA) can be electronically coupled later to optical, genetic, or mass spectrometric results was performed using IC9-2B-S-S-RC-3-4 and IC9-2B-S-S-YC-9 as mass reporters. The electrochemical immunoassays (EC-IA) results were stored electrically, and electronic coupling to additional data required reading mass reporter mass and concentration at the expected values. The MS-IA reagents were added to the sample at the time of EC-IA but were processed after EC-IA analysis, which provided mass spectrometric results sequentially delayed. Mass reporters were measured by LTQ after all of the mass labels were collected from all of the microwells at the same time.

The IC9-2B-S-S-RC-3-4 and IC9-2B-S-S-YC-9 mass reporters produced unique mass spectrometric (MS) signals which are distinct from others and from those mass labels used to make a measurement of MS-IA. The mass reporter concentration was used as an indication of analyte integrity by comparison to acceptance limits. Comparison of observed concentration was within 90% of the expected concentration and indicated the analytes are valid for electronic linking to further analysis of analytes collected by optical, genetic, or mass spectrometric methods. The concentration and detection of mass reporter labels verify the analyte integrity as acceptable and valid for linking to EC-IA results to other associated electronic data (e.g., Sample ID, Patient ID, Sample Data, Patient Data, and the like).

The mass reporter labels (IC9-2B-S-S-RC-3-4 and IC9-2B-S-S-YC-9) were each placed into different wells of specifically defined positions in a sensor array where one well contained an analyte and the other well did not. The detection of the mass reporter labels was able to provide indication of specific microwells that held the mass reporter labels. This allowed identification of an analyte in microwells by the affinity agent added. EC-IA results indicated the presence of the analyte in a microwell and analysis of the mass reporter from that microwell indicated the analyte was suitable for linking to additional data for the analyte captured.

Additional mass reporters with unique masses and concentrations can be added to microwells and used for identification of analytes in a given microwell and to further demonstrate integrity or a lack of damage to an analyte by electrochemical reaction. The expected mass reporter signal and location is known at the time of manufacture and allows the diagnostic system to electronically compare measured mass reporter signals to expected mass reporters while using affinity agent location to identify the analyte and mass signal to verify analyte integrity. The analyte identity and integrity provided by mass reporters can be used for 1) reading additional standardization of mass spectrometric, optical, or PCR analysis; and 2) automatic correction of integrity of an analyte for stability as well other associated factors impacting integrity.

In all examples, the reagents for electrochemical, optical, and mass spectrometric detection could be processed together in one common electrochemical sensor microwell with a size exclusion filter, electrodes, and affinity agents for analyte reagent capture and analyte detection. In all examples, affinity agents allowed the capture of cells, biomolecule analytes, and mass reporters.

In some examples, the affinity agents for analyte capture also allow the capture of a mass reporter used to determine the identity and integrity of the analyte and sample being suitable for additional linking data. In other examples, a mass reporter is used as a marker of microwell location for an analyte released for detection by additional optical, genetic, and/or mass spectrometric analysis. In other examples, a mass reporter is used as an internal standard for an analyte released for detection by additional optical, genetic, and/or mass spectrometric analysis.

While the invention has been illustrated and described in detail in the drawings and foregoing description, the same is to be considered as illustrative and not restrictive in character, it being understood that only the representative embodiments have been shown and described and that all changes, equivalents, and modifications that come within the spirit of the inventions defined by the claims are desired to be protected. All publications, patents, and patent applications cited in this specification are herein incorporated by reference as if each individual publication, patent, or patent application were specifically and individually indicated to be incorporated by reference and set forth in its entirety herein.

1. A method of analysis of complex samples comprising:
introducing an affinity agent with an attached catalyst capable of forming an electrochemical signal; and
measuring the electrochemical signal that is capable of being electronically coupled to optical, genetic, or mass spectrometric results.

2. The method of claim 1, wherein the method contains no interference with optical, genetic, or mass spectrometric detection.

3. The method of claim 1, wherein a mass reporter measures the integrity and identity of an analyte by mass spectrometric detection.

4. The method of claim 1, further comprising processing reagents for electrochemical, optical, and mass spectrometric detection in a common electrochemical sensor microwell with a size exclusion filter, electrodes, and affinity agents for analyte reagent capture and analyte detection.

5. The method of claim 1, wherein mass reporters are not generated upon oxidation at non-reducing voltages of >−0.1.

6. The method of claim 1, wherein the mass reporter causes production of the optical, genetic, or mass spectrometric results as an analyte.

7. The method of claim 1, wherein mass reporter measurements allow electronical coupling of electrochemical analyte detection to optical, genetic, or mass spectrometric results.

8. The method of claim 1, further comprising introducing a catalyst, wherein the catalyst is an enzyme to generate the electrochemical signal.

9. The method of claim 1, wherein an affinity agent contains a fluorescent label for optical detection.

10-13. (canceled)

14. The method of claim 1, wherein mass reporters are generated upon reduction at non-reducing voltages of <−0.1.

15. The method of claim 1, further comprising introducing a catalyst, wherein the catalyst is a nanoparticle to generate the electrochemical signal.

16. The method of claim 1, further comprising of further analyzing analyte by downstream optical methods.

17. The method of claim 1, further comprising of further analyzing analyte by downstream mass spectrometric methods.

18. The method of claim 1, further comprising of further analyzing analyte by downstream immunoassay methods.

19. The method of claim 1, further comprising of further analyzing analyte by downstream genetic method.
